(19) United States
(12) Patent Application Publication
(10) Pub. No.: US 2017/0000302 A1
(43) Pub. Date: Jan. 5, 2017

(54) Urinary device for emergency use

(71) Applicant: Kwok Fai CHEN, N.T. (HK)

(72) Inventor: Kwok Fai CHEN, N.T. (HK)

(21) Appl. No.: 14/789,997

(22) Filed: Jul. 2, 2015

(51) Int. Cl. A47K 11/12; A61F 5/44; A61G 9/00

(52) U.S. Cl. CPC A47K 11/12; A61G 9/006; A61F 5/44

(57) ABSTRACT

An urinary device for emergency use is provided. The urinary device has a base which is provided with a lower opening for resting a buttock. A snap fitting device is also provided on the lower opening for hanging a receptacle.

### BACKGROUND OF THE INVENTION

The present invention belongs to the technical field of devices for urination and defecation, and more specifically relates to an urinary device for emergency use.

Nowadays in human societies, urination and defecation are still private activities which should be done in male or female toilets. The need for urination and defecation cannot be controlled arbitrarily. However, in daily life, such a need may be urgent while a toilet is not available, and this problem is present particularly in some special occasions such as during a long travel on a coach bus or among a special group of people such as patients who cannot walk around freely in the hospital. Therefore, a solution is required to minimize the undesirable physical and psychological impact caused by withholding urine.

### BRIEF SUMMARY OF THE INVENTION

In view of the aforesaid disadvantages now present in the prior art, the present invention provides an urinary device for emergency use. The urinary device has a simple structure facilitating easy use to meet the urgent need for urination and defecation. Therefore, the urinary device is very practical in use.

An urinary device for emergency use is provided to attain the above object. The urinary device comprises a base; the base is provided with a lower opening for resting a buttock; a snap fitting device is provided on the lower opening for hanging a receptacle; the receptacle refers to a plastic bag.

Preferably, an upper cover is provided on an upper end surface of the base, and the upper cover has a shape that matches with the base; an upper opening corresponding to where the lower opening is positioned is also provided on the upper cover for resting the buttock; the upper cover can keep the buttock clean and hygienic during resting of the buttock; when the upper cover is snap-fitted with the base, the plastic bag can also be fastened.

As a modification, one end of the base is connected to one end of the upper cover to facilitate pivot rotation of the upper cover.

Preferably, a side view of the base has an S shape; the S shape facilitates convenient use of the urinary device and also presents the overall structure of the urinary device in a neat and aesthetically pleasing manner,

In actual use, an upper part of the plastic bag is brought upward from a bottom side of the base towards the upper end surface of the base through the lower opening; the upper cover is then snap-fitted to the base to press and fasten the plastic bag so that the plastic bag is hung below the lower opening of the base; after that, a buttock of a user may be rest on the upper opening of the upper cover; a cover object such as a scarf may be used to cover and hide the lower body of the user during urination and defecation; the upper cover is opened again after use and then the plastic bag can be removed from the snap fitting device for subsequent disposal.

The present invention minimizes the undesirable physical and psychological impact caused by withholding urine and relieves the psychological stress of users caused by their realization of the need to withhold urine, thereby promoting the health of the users. The present invention can meet users' need for urination and defecation anytime at anywhere. The present invention has a simple structure facilitating easy use to meet the urgent need for urination and defecation. Therefore, the present invention is very practical in use.

### BRIEF DESCRIPTION OF THE DRAWINGS

FIG. 1 is a structural view of the present invention specifically designed for use by female users.

FIG. 2 is a structural view showing the back of the present invention which is specifically designed for use by female users.

FIG. 3 is a structural view of the present invention specifically designed for use by male users, with its upper cover in an opened condition.

### REFERENCE SIGNS

1: Base; 2: lower opening; 3: snap fitting ring; 4: upper cover; 5 upper opening.

### DETAILED DESCRIPTION OF THE INVENTION

The present invention is further described in detail below with reference to some embodiments and the accompanying drawings. However, the present invention should not be limited to the detailed description herein.

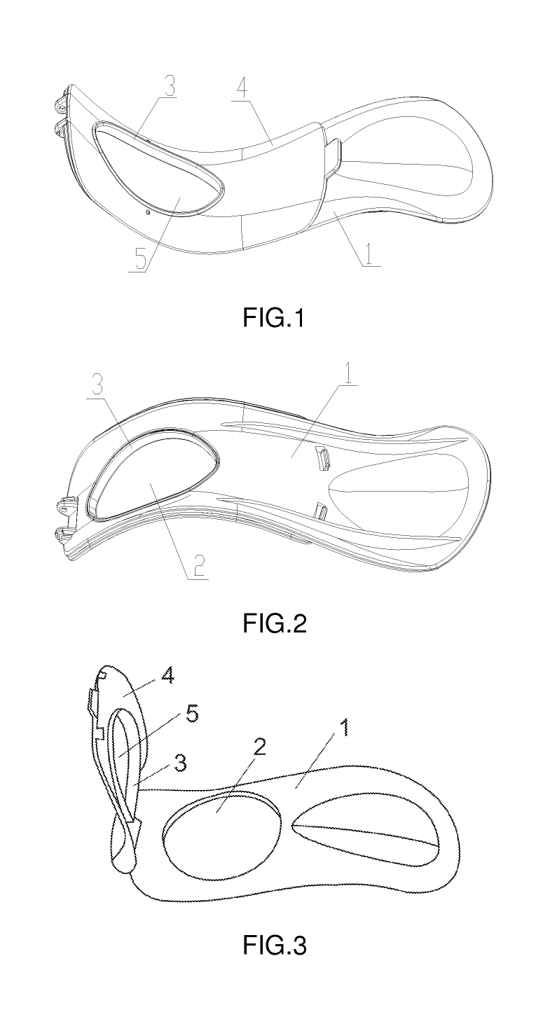

With reference to FIGS. 1 and 2, an urinary device for emergency use according to the present invention comprises a base 1; the base 1 is provided with a lower opening 2 for resting a buttock; a snap fitting device is provided on the lower opening 2 for hanging a receptacle; the receptacle refers to a plastic bag, and the snap fitting device refers to a snap fitting ring 3 disposed around an edge of the lower opening 2; when the base 1 is snap-fitted with an upper cover 4, the upper cover 4 presses against the base 1 to enable to snap fitting ring 3 to hang the plastic bag.

With reference to FIGS. 1 and 2 again, the upper cover 4 is provided on an upper end surface of the base 1, and the upper cover 4 has a shape that matches with the base 1; an upper opening 5 corresponding to where the lower opening 2 is positioned is also provided on the upper cover 4 for resting the buttock; one end of the base 1 is connected to one end of the upper cover 4 to facilitate pivot rotation of the upper cover 4. The upper cover 4 can keep the buttock clean and hygienic during resting of the buttock; when the upper cover 4 is snap-fitted with the base 1, the plastic bag can also be fastened. A side view of the base 1 has an S shape; the S shape facilitates convenient use of the urinary device and also presents the overall structure of the urinary device in a neat and aesthetically pleasing manner.

In actual use, an upper part of the plastic bag is brought upward from a bottom side of the base 1 towards the upper end surface of the base 1 through the lower opening 2; the upper cover 4 is then snap-fitted to the base 1 to press and fasten the plastic bag so that the plastic bag is hung below the lower opening 2 of the base 1; after that, a buttock of a user may be rest on the upper opening 5 of the upper cover 4; a cover object such as a scarf may be used to cover and hide the lower body of the user during urination and defecation; the upper cover 4 is opened again after use and then the plastic bag can be removed from the snap fitting ring 3 for subsequent disposal.

FIG. 3 is a structural view showing the urinary device specifically designed for use by male users, with its upper cover in an opened condition. On the contrary, the urinary device shown in FIGS. 1 and 2 is specifically designed for use by female users. A side view of the base 1 of the urinary device shown in FIG. 3 has an S shape forming a curvature that is different from the curvature of the S shape seen from a side view of the base 1 of the urinary device shown in FIGS. 1 and 2. Also, the snap fitting ring 3 shown in FIG. 3 is provided around an edge of the upper opening 5 of the upper cover 4 instead of being provided around the edge of the lower opening 2.

The present invention minimizes the undesirable physical and psychological impact caused by withholding urine and relieves the psychological stress of users caused by their realization of the need to withhold urine, thereby promoting the health of the users. The present invention can meet users' need for urination and defecation anytime at anywhere. The present invention has a simple structure facilitating easy use to meet the urgent need for urination and defecation. Therefore, the present invention is very practical in use.

A detailed description of the present invention is given above. However, it is obvious that any changes and modification can be made by a person skilled in this field of art without deviating from the scope of the present invention defined by the claims.

What is claimed is:

1. An urinary device for emergency use, comprising a base; the base is provided with a lower opening for resting a buttock; a snap fitting device is provided on the lower opening for hanging a receptacle.

2. An urinary device for emergency use as described in claim 1, wherein an upper cover is provided on an upper end surface of the base; the upper cover has a shape that matches with the base; an upper opening corresponding to where the lower opening is positioned is provided on the upper cover for resting the buttock.

3. An urinary device for emergency use as described in claim 2, wherein one end of the base is connected to one end of the upper cover.

4. An urinary device for emergency use as described in claim 1, wherein a side view of the base has an S shape.
